(19) United States
(12) Patent Application Publication
(10) Pub. No.: US 2024/0424058 A1
(43) Pub. Date: Dec. 26, 2024

(54) COMPOSITION FOR PREVENTING, TREATING OR AMELIORATING POOR OVARIAN RESPONSE SYNDROME COMPRISING RGMC

(71) Applicant: CHA UNIVERSITY INDUSTRY-ACADEMIC COOPERATION FOUNDATION, Pocheon-si (KR)

(72) Inventors: Ji Hyung CHUNG, Seoul (KR); Jae Ho LEE, Yongin-si (KR); Se Hee LEE, Incheon (KR); Yu Jin KIM, Seoul (KR); Myung Joo KIM, Seoul (KR)

(73) Assignee: CHA UNIVERSITY INDUSTRY-ACADEMIC COOPERATION FOUNDATION, Pocheon-si (KR)

(21) Appl. No.: 18/686,643

(22) PCT Filed: Aug. 26, 2022
(86) PCT No.: PCT/KR2022/012826

§ 371 Date: Apr. 2, 2024

(30) Foreign Application Priority Data
Aug. 26, 2021 (KR) 10-2021-0113412

(51) Int. Cl. A61K 38/17; A61P 15/08

(52) U.S. Cl. CPC A61K 38/177; A61P 15/08

(57) ABSTRACT

A method of preventing or treating poor ovarian response syndrome, including administering a pharmaceutical composition containing an RGMc-derived protein. A pharmaceutical composition for preventing, treating or ameliorating infertility, containing an RGMc-derived protein, where the infertility is infertility due to poor ovarian response syndrome. A pharmaceutical composition for promoting pregnancy, containing an RGMc-derived protein.

### TECHNICAL FIELD

This application is based on and claims priority to Korean Patent Application No. 10-2021-0113412, filed on Aug. 26, 2021, the disclosure of which is incorporated by reference herein in its entirety.

The present invention relates to a composition for preventing, treating or ameliorating poor ovarian response syndrome, including an RGMc-derived protein.

### BACKGROUND ART

Currently, there is no distinct treatment method for poor ovarian response (POR) syndrome and infertility caused thereby. Poor ovarian response syndrome is observed in 9% to 24% of infertile female patients and is closely related to low pregnancy success rate. At the European Society of Human Reproduction and Embryology (ESHRE) held in Bologna, Italy in 2010, the diagnostic criteria for poor ovarian response syndrome were presented as follows (diagnosed as poor ovarian response group if 2 or more of the following 3 criteria are met): 1) Old age (40 years or older), 2) 3 eggs or fewer are released when collecting eggs according to the controlled ovarian stimulation (COS) protocol, 3) Antral Follicle Count (AFH) is less than 5 to 7 or anti-Mullerian hormone (AMH) level is less than 0.5 ng/ml to 1.1 ng/ml. In this regard, methods such as hypostimulation therapy, use of natural cycles, and high-dose ovulation induction injection are used to induce controlled ovarian stimulation, but it is very difficult to apply such COS protocols to patients who have a very low hormonal response or do not respond appropriately to the inducing hormone. Therefore, the development of a novel therapeutic agent that may be applied to patients with poor ovarian response syndrome is required.

Meanwhile, neogenin is a multifunctional receptor belonging to the immunoglobulin superfamily and has been reported to be abundantly expressed in the ovaries. Neogenin has three main signal ligands: repulsive guidance molecules (RGMs) including RGMa, RGMb and RGMc; netrin-1; and slit. These ligands play different roles each other through separate pathways depending on the tissue and organ, in various processes such as neurogenesis, mammalian gland formation, and apoptosis pathways. However, nothing is yet known about the role of neogenin ligands or their signaling pathways in follicular development.

### DISCLOSURE

### Technical Problem

It was confirmed by the present inventors that neogenin is expressed at all stages of follicular development, and that when treating ovaries with RGMc among the signaling ligands of neogenin, RGMc is involved in both gonadotropin-independent and gonadotropin-dependent follicular development processes. Furthermore, it was confirmed that the RGMc induces normal development of the ovaries by influencing from the early growth phase of the follicle to the late ovulation phase, thereby restoring ovarian functions in patients with poor ovarian response syndrome to be used for infertility treatment, thus the present invention was completed.

An aspect is to provide a pharmaceutical composition for preventing, treating or ameliorating poor ovarian response syndrome, including an RGMc-derived protein.

Another aspect is to provide a health functional food composition for preventing or improving poor ovarian response syndrome, including an RGMc-derived protein.

Another aspect is to provide a pharmaceutical composition for preventing, treating or ameliorating infertility including an RGMc-derived protein, and to provide a health functional food composition for preventing or ameliorating infertility including an RGMc-derived protein.

Another aspect is to provide a pharmaceutical composition for promoting pregnancy including an RGMc-derived protein, and to provide a health functional food composition for preventing or ameliorating including an RGMc-derived protein.

### Technical Solution

An aspect is to provide a pharmaceutical composition for preventing, treating or ameliorating poor ovarian response syndrome, including an RGMc-derived protein.

As used herein, the term “repulsive guidance molecule (RGM)” refers to one of the signal ligands of Neogenin, and the repulsive guidance molecule is composed of a gene family consisting of three different genes, RGMa, RGMb, and RGMc. The RGMc (Repulsive guidance molecule C) may be composed of the nucleotide sequence of SEQ ID NO: 1 or SEQ ID NO: 3, and the RGMc protein may be composed of the amino acid sequence of SEQ ID NO: 2 or SEQ ID NO: 4. In an embodiment, the mouse RGMc gene (Gene ID: 69585) may be composed of the nucleotide sequence of SEQ ID NO: 1, and the mouse RGMc protein may be composed of the amino acid sequence of SEQ ID NO: 2. In an embodiment, the human RGMc gene (Gene ID: 148738) may be composed of the nucleotide sequence of SEQ ID NO: 3, and the human RGMc protein may be composed of the amino acid sequence of SEQ ID NO: 4.

As used herein, the genes may include a base sequence in which one or more bases are deleted, substituted, or inserted in the base sequence of SEQ ID NO: 1, and the proteins not only refer to a protein composed of amino acids of SEQ ID NO: 2 or a fragment thereof, but also may include mutations such as one or more substitutions, insertions, deletions, etc. in these proteins. In an embodiment, the RGMc may not only be a wild-type RGMc, but also be an RGMc-derived protein including the amino acid sequence of SEQ ID NO: 2, SEQ ID NO: 4, SEQ ID NO: 31, SEQ ID NO: 32, SEQ ID NO: 33, or SEQ ID NO: 34, and may be selected from the group of proteins having amino acid sequences represented by SEQ ID NO: 2, SEQ ID NO: 4, SEQ ID NO: 31, SEQ ID NO: 32, SEQ ID NO: 33, and SEQ ID NO: 34, but is not limited thereto. The term ‘RGMc-derived’ means including not only a wild-type RGMc protein or gene, but also all specific parts of the protein or gene.

As used herein, it is clear to those skilled in the art that the sequences of the RGMc gene and protein are only examples and are not limited thereto. Sequences having substantial sequence identity or substantial sequence homology to the above sequences are also included within the scope of the present invention. As used herein, the terms “substantial sequence identity” or “substantial sequence homology” mean that a sequence exhibits substantial structural or functional identity with another sequence.

The RGMc is known to bind to neogenin, but the role of RGMc in follicle development or its signaling pathway is still unknown.

As used herein, the term “poor ovarian response (POR) syndrome” is also used interchangeably with “ovarian hyporesponse” or “ovarian hypofunction,” and as described above, refers to a case diagnosed as poor ovarian response syndrome according to the Bologna criteria.

As used herein, the term “preventing” may refer to any action that suppresses or delays poor ovarian response syndrome by administering the composition, “treating” may refer to any action that improves or beneficially changes the symptoms of poor ovarian response syndrome by administering the composition, and “ameliorating” may refer to any action that at least reduces parameters related to the condition being treated, for example, the degree of symptoms.

The pharmaceutical composition according to an aspect may include 0.0001 wt % to 50 wt %, 0.001 wt % to 50 wt %, 0.01 wt % to 50 wt %, 0.1 wt % to 50 wt %, 0.0001 wt % to 25 wt %, 0.001 wt % to 25 wt %, 0.01 wt % to 25 wt %, 0.0001 wt % to 10 wt %, 0.001 wt % to 10 wt %, 0.01 wt % to 10 wt %, or 0.1 wt % to 10 wt % of RGMc protein, which is an active ingredient, based on the total weight of the composition.

The pharmaceutical composition may include other ingredients, in addition to RGMc, within the range that does not impair the intended main effect, preferably that have a synergistic effect on the main effect.

In addition, the pharmaceutical composition may further include a pharmaceutically acceptable carrier, excipient, and diluent in addition to the active ingredient described above for administration.

For example, lactose, textose, sucrose, sorbitol, mannitol, xylitol, erythritol, maltitol, starch, gum acacia, alginate, gelatin, calcium phosphate, calcium silicate, cellulose, methylcellulose, amorphous cellulose, polyvinyl pyrrolidone, water, methylhydroxybenzoate, propylhydroxybenzoate, talc, magnesium stearate, or mineral oil may be used as a carrier, excipient and diluent, and a target organ-specific antibody or other ligands may be used in combination with the carrier to specifically act on the target organ.

The pharmaceutical composition may be prepared in various parenteral or oral administration forms according to known methods. Representative formulations for parenteral administration may include aerosol formulations and injectable formulations.

Solid preparations for oral administration include tablets, pills, powder, granules, capsules, etc., and such solid preparations are prepared by mixing the active ingredient with at least one excipient, such as starch, calcium carbonate, sucrose or lactose, and gelatin. Additionally, in addition to simple excipients, lubricants such as magnesium stearate and talc may also be used.

Liquid preparations for oral administration include suspensions, oral solutions, emulsions, syrups, etc., and in addition to commonly used simple diluents such as water and liquid paraffin, various excipients such as wetting agents, sweeteners, fragrances, and preservatives may be included.

Preparations for parenteral administration include sterile aqueous solutions, non-aqueous solutions, suspensions, emulsions, lyophilized preparations, and suppositories. Propylene glycol, polyethylene glycol, vegetable oils such as olive oil, and injectable esters such as ethyl oleate may be used as non-aqueous solvents and suspensions. Wethepsol, macrogol, Tween 61, cacao butter, laurin butter, glycelogelatin, etc. may be used as a base for suppositories. However, it is not limited thereto, and the pharmaceutical composition according to an aspect may be formulated according to the purpose using any method known in the art without limitation.

The pharmaceutical composition may be administered in a pharmaceutically effective amount.

As used herein, the term “pharmaceutically effective amount” refers to an amount sufficient to treat a disease with a reasonable benefit/risk ratio applicable to medical treatment, and the effective dose level may be determined based on the factors including the type and severity of the individual, age, gender, activity of the medications, sensitivity to the medications, time of administration, route of administration and excretion rate, duration of treatment, medications used simultaneously, and other factors well known in the medical field. The composition may be administered as an individual therapeutic agent or in combination with other therapeutic agents, and may be administered sequentially or simultaneously with conventional therapeutic agents. And it may be administered single or multiple times. Considering all of the above factors, it is important to administer a minimum amount that may achieve maximum effect without side effects, and such amount may be easily determined by a person skilled in the art.

In the pharmaceutical composition according to an aspect, the RGMc may increases at least one expression selected from the group consisting of Oct3/4, Nanog, and p63,
increase at least one expression selected from the group consisting of Ptgs1, Edn2 and Hpgds, and/or
reduce at least one expression selected from the group consisting of Tbxa2r, Oxtr, and Adra1d.

In an example, 0.5 mg/kg of RGMc was injected into mice, and 75 IU of pregnant mare serum gonadotropin (PMSG) was injected 12 hours later, and it was confirmed that the RGMc may promote the development of ovarian follicles through both gonadotropin-independent and gonadotropin-dependent processes (FIGS. 2 and 3).

Specifically, it was confirmed that the RGMc increases the expression of Oct3/4, Nanog, and p63 in the gonadotropin-independent process of follicular development, and increases the expression of Ptgs1, Edn2, and Hpgds in the gonadotropin-dependent process of follicular development, and reduces the expression of Tbxa2r, Oxtr, and Adra1d, thereby promoting ovarian follicle development and at the same time suppressing ovulation and promoting normal follicle development (FIGS. 5, 6, and 9).

Another aspect is to provide a health functional food composition for preventing or ameliorating poor ovarian response syndrome, including an RGMc-derived protein.

In the health functional food composition, parts that overlap with the description of an RGMc-derived protein and poor ovarian response syndrome are omitted.

The health functional food may be used simultaneously or separately with medications for treatment before or after the onset of poor ovarian response syndrome in order to prevent or ameliorate poor ovarian response syndrome.

In the health functional food, the active ingredient may be added directly to food or used together with other food or food ingredients, and may be used appropriately according to the conventional methods. The mixing amount of the active ingredient may be appropriately determined depending on the purpose of use (for preventing or ameliorating). In general, when preparing food or beverage, the health functional food may be added in an amount of about 15 wt % or less, more specifically about 10 wt % or less, based on the raw material. However, in the case of long-term intake for the purpose of health and hygiene or health control, the amount may be below the above range.

The health functional food may further include at least one of diluents, excipients or additives such as carriers, fillers, extenders, binders, wetting agents, disintegrants, and surfactants, and it may be formulated as one selected from the group consisting of tablet, pill, powder, granule, powder, capsule and liquid formulations. The food that may be added include various foods, powder, granule, tablet, capsule, syrup, beverage, gum, tea, vitamin complex, health functional foods, etc.

In addition to the above, the health functional food according to an aspect may include various nutrients, vitamins, minerals (electrolytes), flavoring agents such as synthetic and natural flavors, colorants and thickening agents (cheese, chocolate, etc.), pectic acid and salts thereof, alginic acid and salts thereof, organic acids, protective colloidal thickeners, pH adjusters, stabilizers, preservatives, glycerin, alcohol, carbonating agents used in carbonated beverages. These components may be used independently or in combination, and the proportions of these additives may also be appropriately selected by those skilled in the art.

Another aspect is to provide a method of treating poor ovarian response syndrome including administering the composition to an individual having poor ovarian response syndrome.

The individual may be a mammal including cows, pigs, sheep, chickens, dogs, humans, or the like, and individuals whose poor ovarian response syndrome is treated by the administration of the composition may be included without limitation.

In this regard, the composition may be administered in a single or multiple dose in a pharmaceutically effective amount. In this regard, the composition may be administered in the form of liquid, powder, aerosol, capsule, enteric-coated tablet or capsule, or suppository. Routes of administration include, but are not limited to, intraperitoneal administration, intravenous administration, intramuscular administration, subcutaneous administration, endothelial administration, oral administration, topical administration, intranasal administration, intrapulmonary administration, and intrarectal administration. However, when administered orally, since peptides are digestible, oral compositions must be formulated to coat the active agent or protect it from degradation in the stomach.

The pharmaceutical composition may be administered in a pharmaceutically effective amount, for example, from 0.0001 mg/kg to 100 mg/kg, 0.001 mg/kg to 10 mg/kg, 0.01 mg/kg to 10 mg/kg, or 0.1 mg/kg to 10 mg/kg, but it is not limited thereto. The dosage may vary depending on the patient's age, gender, weight, health condition, diet, administration period, administration method, or severity of the disease.

Another aspect is to provide a pharmaceutical composition for preventing, treating or ameliorating infertility including an RGMc-derived protein, and to provide a health functional food composition for preventing or ameliorating infertility including an RGMc-derived protein.

Regarding the composition, parts that overlap with the description of the RGMc-derived protein, the pharmaceutical composition, and the health functional food composition are omitted.

As used herein, the term “infertility” refers to a condition in which a couple (including those in a de facto marriage) are unable to become pregnant even after one year despite having a normal sexual life without contraception.

In this specification, the infertility refers to female infertility, and may be, for example, infertility due to poor ovarian response (POR) syndrome.

According to an embodiment, the RGMc is involved in all processes from the early growth phase of the follicle to the late ovulation phase to induce normal development of the ovaries, thereby preventing, treating, or ameliorating infertility by restoring the function of aged or less reactive ovaries.

The composition may include RGMc alone, or may further include substances effective in preventing, treating, or ameliorating infertility as active ingredients. In addition to the active ingredients, the composition may further include additional ingredients, namely, pharmaceutically acceptable or nutritionally acceptable carriers, excipients, diluents or accessory ingredients, depending on the formulation, method of use, and purpose of use.

The composition may be used alone to prevent, treat or ameliorate infertility, or may be used in combination with surgery, hormone treatment, chemical treatment and biological response regulators, etc.

Another aspect is to provide a pharmaceutical composition for promoting pregnancy including an RGMc-derived protein, and to provide a health functional food composition for preventing or ameliorating, including an RGMc-derived protein.

Regarding the composition, parts that overlap with the description of the RGMc-derived protein, infertility, pharmaceutical composition, and health functional food composition are omitted.

As used herein, the term “pregnancy” refers to a process in which a fertilized egg implants into the uterine lining and develops into a fetus while receiving nutrition from the mother.

According to an embodiment, the RGMc is involved in all processes from the early growth phase of the follicle to the late ovulation phase and induces normal development of the ovaries, thereby increasing the possibility of pregnancy in infertile patients.

The composition may include RGMc alone or may further include a substance effective in promoting pregnancy as an active ingredient, and in addition to the active ingredient, and the composition may further include additional ingredients, that is, pharmaceutically acceptable or nutritionally acceptable carriers, excipients, diluents or auxiliary ingredients depending on the formulation, method of use, and purpose of use.

The composition may be used alone to promote pregnancy, or may be used in combination with surgery, hormone treatment, chemical treatment and biological response regulators, etc.

Another aspect is to provide a method of preventing or treating poor ovarian response syndrome, including administering a pharmaceutical composition including the RGMc-derived protein.

Another aspect is to provide a method of preventing or treating poor ovarian response syndrome with a composition including the RGMc-derived protein.

### Advantageous Effects

When the composition according to an aspect is used, this may induce the normal development of the ovarian with poor response so as to treat poor ovarian response syndrome and infertility caused thereby, and may effectively recover aged ovarian functions to increase the possibility of pregnancy.

### DESCRIPTION OF DRAWINGS

FIG. 1 is a diagram showing the results of determining the expression profile of neogenin in ovaries. (a) Immunostaining results using anti-neogenin antibody. Neogenin (green), p63 (red) and DAPI (blue). Arrows indicate follicles with positive neogenin staining. (b) Results of confocal image analysis of mouse oocytes. Neogenin (red), DAPI (blue). GV: Germinal Vesicle, MII: Metaphase II, BF: Bright Field. (c) Results of RT-PCR analysis of neogenin mRNA in mouse oocytes. GAPDH was used as an internal control. (d) Results of western blot of neogenin protein in mouse ovary. Beta-actin was used as an internal control. Neo: Neogenin.

FIG. 2 is a diagram showing the results of treating mouse ovaries stimulated with PMSG with RGMc and determining the development pattern of ovarian follicles. (A) H&E staining results (Scale bar=200 μm). (B) Total number of follicles in control (CTL) and RGMc treated group. (C) Number of antral follicles. (D) Number of collected oocytes.

FIG. 3 is a diagram analyzing mRNA and protein expression in RGMc-treated ovaries. (A) RT-PCR analysis results. (B) Quantitative real-time PCR analysis results. (C) Western blot analysis results.

FIG. 4 is a diagram showing the NGS results of whole transcriptomes of RGMc-treated ovaries. (A) Venn diagram of genes with up-/down-regulated expression levels (fold-change >4) in control and RGMc-treated ovaries. A total of 24,255 genes were used for RNA-sequencing analysis. (B) Cluster map of genes with up-/down-regulated expression levels (fold-change >4).

FIG. 5A is a diagram confirming the relationship between up-/down-regulated genes that showed an absolute fold-change of 4-fold or more in RGMc-treated ovaries, and is a Heatmap based on significantly enriched terms and up-/down-expressed genes in RGMc-treated ovaries. Blue and red colors in the top and left sidebars indicate up-/down-expressed terms in RGMc-treated ovaries, respectively, and black color indicates non-satisfied terms that were enriched by 60% or more in up-/down-expressed genes. Red and green colors in the Heatmap indicate the presence/absence of genes, respectively.

FIGS. 5B and 5C are diagrams confirming the relationship between up-/down-regulated genes that showed an absolute fold-change of 4-fold or more in RGMc-treated ovaries, and are about network-based analysis between significantly enriched terms and up-/down-expressed (fold-change >4) genes. Dotted circles and hatched circles represent up-/down-expressed genes/terms in RGMc-treated ovaries, respectively, and open circles represent non-satisfied terms that were enriched by 60% or more in up-/down-expressed genes. White, grid arrows indicate target genes for feasibility analysis that were up-/down-expressed according to the RGMc treatment.

FIG. 6 is a diagram showing the results of quantitative real-time PCR analysis to verify NGS data of RGMc-treated ovaries. (A) RT-PCR analysis results. (B) Quantitative real-time PCR analysis results. *p<0.05 vs. control.

FIG. 7 is a diagram showing the results of analyzing PGD2 levels in the follicular fluid of normal control and POR patients.

FIG. 8 is a diagram showing ELISA results of follicular fluid and cumulus cell culture medium according to RGMc treatment. (A) Neogenin gene expression levels in patients with normal ovarian response (NOR) and patients with POR (POR). (B, C) PGD2 levels in cumulus cell culture medium. *p<0.05 vs. Young and old groups with normal ovarian response.

FIG. 9 is a diagram showing a mechanism for promoting follicular development of a composition according to an aspect.

FIG. 10 is a diagram showing the structures of four novel proteins derived from RGMc protein according to an aspect.

FIG. 11 is a diagram confirming the level of PGD2 while treating cumulus cells with the four novel proteins, the control, and total RGMc proteins, respectively.

FIG. 12 is results of determining the levels of ovarian function-related genes by PCR while treating cumulus cells with each of the four novel proteins.

### BEST MODE

Hereinafter, the present invention will be described in more detail through examples. However, these examples are for illustrative purposes only and the scope of the present invention is not limited to these examples.

### Experimental Method

### 1. Preparation of Test Animals, and RGMc Protein and Injection of RGMc

6-8 week old ICR mice (Oriental Bio, Korea) weighing 20-25 g were used as test animals, and a total of 60 mice, 30 in the control group and 30 in the experimental group, were used with the approval of the Animal Experiment Ethics Committee of Cha University Medical School. For in vivo hormone stimulation, 0.5 mg/kg RGMc was injected into the experimental group through intraperitoneal injection, and 12 hours later, 75 IU pregnant mare serum gonadotropin (PMSG) was injected through intraperitoneal injection. For the control group, only 75 IU PMSG was injected. After the mice were euthanized by CO₂ gas inhalation in a chamber, both ovaries were collected, one of which was fixed with 4% paraformaldehyde overnight at room temperature for histological analysis, and the other was stored at −80° C.

### 2. Histological Analysis and Confocal Image Analysis

The expression of neogenin was measured in mouse ovaries and oocytes. First, the ovaries fixed with 4% paraformaldehyde were tissue processed using an automatic tissue processor and then embedded in paraffin to create tissue sections. The first and 50th tissue sections of each ovarian sample were stained with Hematoxylin & Eosin (H&E) and observed under a light microscope to count the number of follicles. Additionally, each tissue was deparaffinized by treatment with xylene, and then rehydrated by sequentially incubating with low to high concentrations of ethanol at room temperature. Tissue sections were reacted with a rabbit anti-neogenin antibody (1:100 dilution, Santa Cruz Biotechnology, USA) and a mouse anti-p63 antibody (1:100 dilution, Santa Cruz Biotechnology, USA), then treated with Alexa Fluor 488- and Alexa Fluor 594-conjugated secondary antibodies (1:200 dilution, Molecular Probes, USA), and were stained with Hoechst 33342 (Thermo Fisher Scientific, USA) for 5 minutes at room temperature to identify the nuclei of cells. After washing with PBS, the tissue sections were mounted with an anti-fade mounting solution (VECTASHIELD H-1000, USA), covered with a cover glass, and the immunostaining results were read under a confocal microscope. In the case of oocytes, they were fixed with 4% paraformaldehyde at room temperature for 10 minutes, then treated with 0.1% Triton-X and washed with PBS for 30 minutes at room temperature. A rabbit anti-neogenin antibody (1:100 dilution in PBS including 0.1% BSA) was added and incubated at 4° C. overnight. Then, the cells were incubated with a secondary antibody (anti-rabbit labeled 555) for 2 hours at room temperature and washed with PBS. Nuclei were stained with Hoechst 33342 for 5 minutes at room temperature, washed twice with PBS, and the stained oocytes were transferred to a dish and the immunostaining results were read under a confocal microscope.

### 3. Gene Expression Analysis

Gene expression in RGMc-treated ovaries was analyzed. Specifically, 100 ng of total RNA was isolated from ovaries using TRIzol according to the manufacturer's protocol, was reverse transcribed into cDNA using AccuPower CycleScript RT Premix (Bioneer, Korea), and then PCR was performed with AccuPower Taq PCR PreMix (Bioneer, Korea) using the cDNA as a template and a set of mouse Neogenin, Oct3/4, Nanog, p63, and beta-actin specific primers. 10 pmol/μL of forward and reverse primers and 200 ng of template cDNA were added to a AccuPower Taq PCR PreMix tube, distilled water was added to be a total volume of 20 μL, and the PCR reaction was performed for a total of 32 cycles, with one cycle of denaturation at 95° C. for 1 minute and at 95° C. for 30 seconds, annealing at 60° C. for 30 seconds, and extension at 72° C. for 50 seconds. After electrophoresis of the PCR product using 2% agarose gel, the results were read by UV irradiation using a WSE-6100 LuminoGraph (ATTO, Japan).

### 4. Protein Expression Analysis

Protein expression in the RGMc-treated ovaries was analyzed by Western blot. Specifically, ovarian tissue samples were homogenized in PRO-PREP™ protein extraction solution (iNtRON Biotechnology, Korea), the proteins included in Laemmli buffer were denatured by heating at 95° C. for 5 minutes, were electrophoresed using 10% SDS-PAGE, and were transferred to a 0.2 μm nitrocellulose membrane (Bio-Rad, USA). The transferred membrane was blocked with TBST buffer including 5% BSA for 1 hour at room temperature, and then incubated with a rabbit anti-neogenin polyclonal (1:2000 dilution, Novus, USA), which is a primary antibody diluted in TBST buffer including 1% BSA, a rabbit anti-Oct3/4 (1:2000 dilution, Santa Cruz Biotechnology, USA), a mouse anti-Nanog (1:2000 dilution, Santa Cruz Biotechnology, USA), a mouse anti-p63 (1:2000 dilution, Santa Cruz Biotechnology, USA), and a mouse anti-beta-actin (1:2000 dilution, Invitrogen, USA) at 4° C. overnight. After washing with TBST buffer, the membrane was reacted with a horseradish peroxidase-conjugated anti-mouse and anti-rabbit IgG secondary antibodies (Bio-Rad). Protein bands were visualized using Clarity™ Western ECL Substrate (Bio-Rad) and WSE-6100 LuminoGraph.

### 5. NGS Analysis

The whole transcriptomes of the control and experimental group ovaries were analyzed through NGS (Next Generation Sequencing). Specifically, total RNA was isolated from ovaries using TRIzol according to the manufacturer's protocol, and the purified mRNA was used for NGS. Sequencing was performed on the mRNA using a MiSeq sequencer (Illumina, USA), and NGS data was analyzed using the REVIGO program and Gene Ontology. Next, differentially expressed gene (DEG) and enrichment analyzes were performed using the samples from four mice (Mus musculus) per group. Raw sequencing data was mapped to perform transcriptome assembly using HISAT2 (ver. 2.1.0), Bowtie2 (ver. 2.3.4.1), and StringTie (ver. 1.3.4d). The aligned reads count was normalized using TMM (Trimmed Mean of M-values) method, genes that showed an absolute 4-fold or greater expression change were extracted, and agglomerative clustering analysis was performed using Euclidean and Ward algorithms. A database including 20,196 reference genes was used for gene enrichment analysis. Using ClueGO (ver. 2.5.5) module of Cytoscape (ver. 3.7.2), the pathways of each gene whose expression levels were up-/down-regulated in RGMc-treated ovaries were analyzed. A two-sided hypergeometric test was performed using Bonferroni step-down correction to determine the statistical significance. For each gene cluster with up-/down-regulated expression levels, GO terms with an enrichment percentage exceeding 60% were selected. The relationship between terms was determined based on the Kappa score.

### 6. RT-qPCR

RT-qPCR was performed to validate NGS data obtained from the control and the RGMc-treated ovaries. Based on the KEGG pathway, primers targeting up-regulated genes (Ptgs1, Edn2, and Hpgds) and down-regulated genes (Tbxa2r, Oxtr, and Adra1d) were selected (see, Table 1 below).

TABLE 1

| Gene | | Primer sequence (5′-3′) | |
|---|---|---|---|
| Neogenin | Neogenin | Forward | GTATGTCGCCTCGCT |
| | | direction | ACCTG |
| | | (SEQ ID | |
| | | NO: 5) | |
| | | Reverse | GCCACAGAGAAGTCA |
| | | direction | TCGGA |
| | | (SEQ ID | |
| | | NO: 6) | |
| Oct3/4 | Octamer- | Forward | CACGAGGTGGAAAGC |
| | binding | direction | AACTCA |
| | transcription | (SEQ ID | |
| | factor 4 | NO: 7) | |
| | | Reverse | AGATGGTGGTCTGGC |
| | | direction | TGAAC |
| | | (SEQ ID | |
| | | NO: 8) | |
| Nanog | Nanog | Forward | CACCCACCCATGCTA |
| | | direction | GTCTT |
| | | (SEQ ID | |
| | | NO: 9) | |
| | | Reverse | ACCCTCAAACTCCTG |
| | | direction | GTCCT |
| | | (SEQ ID | |
| | | NO: 10) | |
| p63 | p63 | Forward | GTATCGGACAGCGCA |
| | | direction | AAGAACG |
| | | (SEQ ID | |
| | | NO: 11) | |
| | | Reverse | CTGGTAGGTACAGCA |
| | | direction | GCTCATC |
| | | (SEQ ID | |
| | | NO: 12) | |
| Ptgs1 | Prostaglandin- | Forward | CCTCGACAACTACCA |
| | endoperoxide | direction | GTGTG |
| | synthase 1 | (SEQ ID | |
| | | NO: 13) | |
| | | Reverse | CACAAATTCCCAGAG |
| | | direction | CCAGT |
| | | (SEQ ID | |
| | | NO: 14) | |
| Edn2 | Endothelin 2 | Forward | GACCTCTCTCCGAAA |
| | | direction | GCTGAG |
| | | (SEQ ID | |
| | | NO: 15) | |
| | | Reverse | CCGTTTCCTCCTGTC |
| | | direction | TCCAC |
| | | (SEQ ID | |
| | | NO: 16) | |
| hpgds | Hematopoietic | Forward | GGACACGCTGGATGA |
| | | direction | CTTCA |
| | | (SEQ ID | |
| | | NO: 17) | |
| | Prostaglandin | Reverse | TCCCAGTAGAAGTCT |
| | D Synthase | direction | GCCCA |
| | | (SEQ ID | |
| | | NO: 18) | |
| Oxtr | Oxytocin | Forward | GTTCTCACCATCCTC |
| | Receptor | direction | GGCA |
| | | (SEQ ID | |
| | | NO: 19) | |
| | | Reverse | CTAACCAGCCCAAGG |
| | | direction | ACAGG |
| | | (SEQ ID | |
| | | NO: 20) | |
| Tbxa2r | Thromboxane | Forward | TCGGGCTCATATTCG |
| | receptor | direction | CACTC |
| | | (SEQ ID | |
| | | NO: 21) | |
| | | Reverse | AACCATCATCTCCAC |
| | | direction | CTCGC |
| | | (SEQ ID | |
| | | NO: 22) | |
| Andra1d | Adrenergic | Forward | TTCTTCGTCCTGTGC |
| | receptor, | direction | TGGTTC |
| | alpha 1d | (SEQ ID | |
| | | NO: 23) | |
| | | Reverse | AGATGAGCGGGTTCA |
| | | direction | CACAG |
| | | (SEQ ID | |
| | | NO: 24) | |
| beta- | beta-actin | Forward | CATTGCTGACAGGAT |
| actin | | direction | GCAGAAGG |
| | | (SEQ ID | |
| | | NO: 25) | |
| | | Reverse | TGCTGGAAGGTGGAC |
| | | direction | AGTGAGG |
| | | (SEQ ID | |
| | | NO: 26) | |

100 ng of total RNA was isolated from ovaries using TRIzol according to the manufacturer's protocol, was reverse transcribed into cDNA using AccuPower CycleScript RT Premix (Bioneer, Korea), and then qPCR was performed with the cDNA as a template using SsoAdvanced Universal SYBR Green Supermix (Bio-Rad, USA). Specifically, 10 pmol/μL of forward and reverse primers, 200 ng of template cDNA, and 2× SsoAdvanced Universal SYBR Green Supermix were added to the tube, distilled water was added to be a total volume of 20 μL, and the PCR reaction was performed for a total of 40 cycles, with one cycle of denaturation at 95° C. for 3 minutes and at 95° C. for 10 seconds, annealing at 60° C. for 30 seconds, and extension at 72° C. for 20 seconds. The expression of each gene was normalized to beta-actin. All experiments were performed in triplicate.

### 7. Statistical Analysis

In all experiments, data were expressed as mean±standard error (SEM) of the triplicate experiments, and statistical comparisons were analyzed by one-way ANOVA combined with Bonferroni test. p values of <0.05 (*), <0.01 (**), and <0.001 (***) were considered as statistically significant.

### EXPERIMENT RESULTS

### Example 1. Expression Analysis of Neogenin

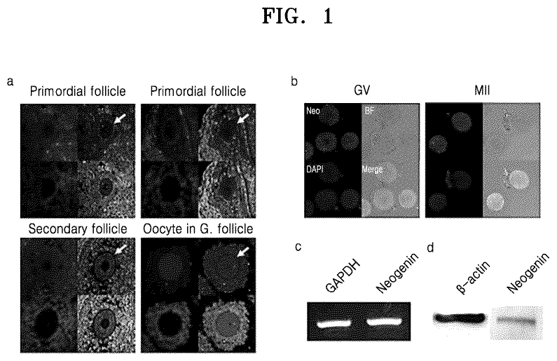

The expression of neogenin was analyzed in mouse ovaries and oocytes, and the results are shown in FIG. 1. As shown in FIG. 1, it was confirmed that neogenin was expressed at all stages of follicular development. Specifically, neogenin was expressed in all of a primordial follicle, a primary follicle, a secondary follicle, and a graafian follicle (FIG. 1A), and neogenin stained with an anti-neogenin antibody (red) was identified in immature oocytes in Germinal Vesicle (GV) and in mitotic Metaphase II (MII) (FIG. 1B). In addition, it was confirmed that the same results were obtained in the mRNA and protein expression analysis of mouse ovaries (FIGS. 1C and 1D).

The above results showed that neogenin is expressed at all stages of follicular development, and the treatment of neogenin ligand may be involved in both gonadotropin-independent and gonadotropin-dependent processes of follicular development.

### Example 2. Analysis of Follicle Development According to RGMc Treatment

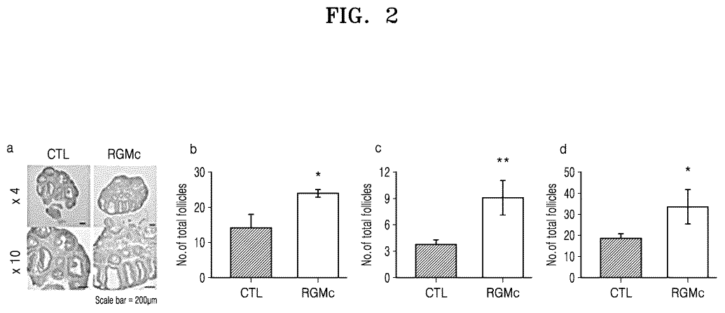

PMSG was injected into mice for in vivo hormonal stimulation, and the effect of RGMc treatment on the ovaries was determined through histological analysis. As shown in FIG. 2, as a result of H&E staining, it was confirmed that the total number of follicles, including preantral follicles and antral follicles, was significantly increased compared to the control group (FIG. 2B), and among them, the number of antral follicles was 2.5 times higher than in the control group (FIG. 2C), that about twice as many oocytes were collected in RGMc-treated ovaries compared to the control ovaries (FIG. 2D).

Through the above results, it was confirmed that treatment with RGMc, a neogenin ligand, may promote ovarian follicle development through both gonadotropin-independent and gonadotropin-dependent processes.

### Example 3. Analysis of Expression Changes of Genes and Proteins Related to Follicular Development

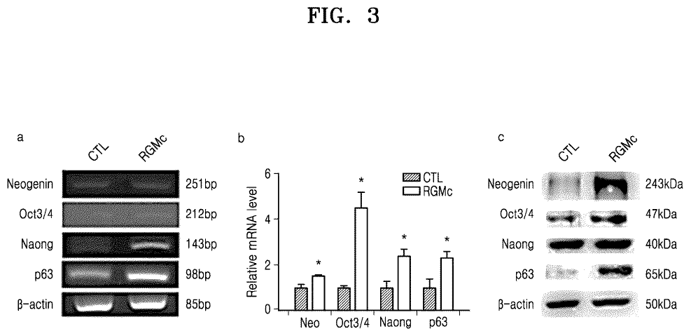

The mRNA expression patterns of the primordial follicle factors Oct3/4, Nanog, p63, and Neogenin were analyzed through RT-PCR. As shown in FIG. 3, it was confirmed that the mRNA expression levels of Oct3/4, Nanog, p63, and Neogenin were significantly increased in the RGMc-treated group compared to the control group (FIG. 3A). In particular, the mRNA expression levels of Oct3/4, Nanog, and p63 increased about two-fold compared to the control group (FIG. 3B). In addition, consistent with the gene expression analysis results, protein expression analysis also found that the protein expression levels of Oct3/4, Nanog, p63, and Neogenin were significantly increased in the RGMc-treated group compared to the control group (FIG. 3C).

Through the above results, it was confirmed that the treatment with RGMc increases the activity of primordial follicles. In addition, given that Oct3/4, Nanog, and p63 regulate the proliferation of stromal cells and the differentiation of granulosa cells into prefollicles, it was confirmed that the treatment with RGMc upregulates the expression levels of Oct3/4 and Nanog to promote COS, thereby improving the survival of primordial follicles in the ovaries. In addition, p63 is a regulator of meiosis and plays an important role in cell cycle control of primordial follicles, so it was confirmed that the RGMc treatment may promote follicle development in the ovaries.

### Example 4. Whole Transcriptome and Gene Enrichment Analysis

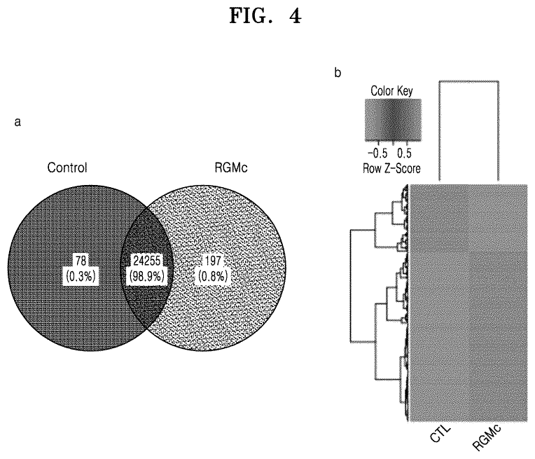

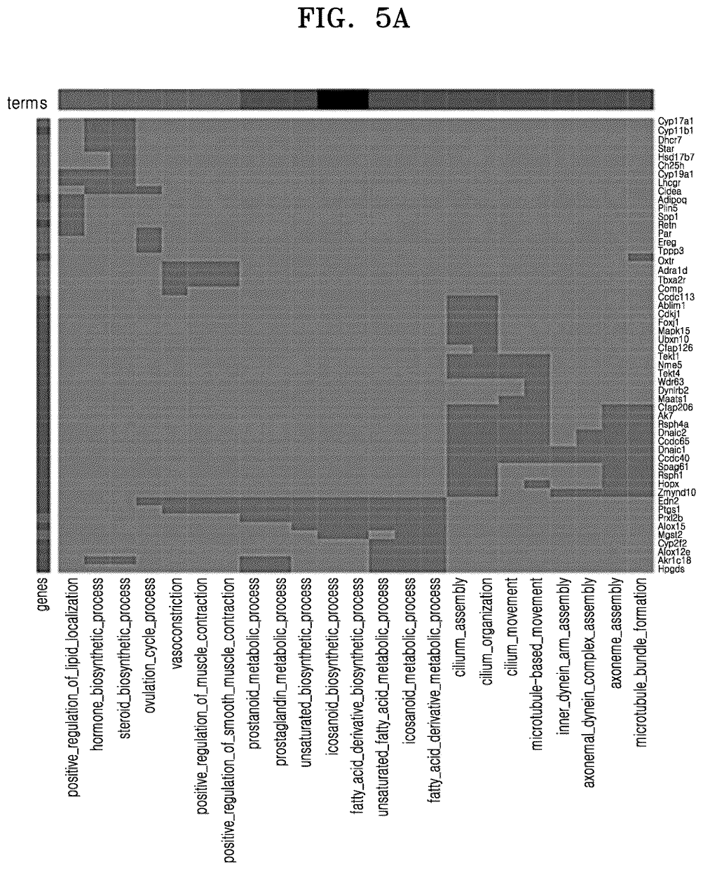

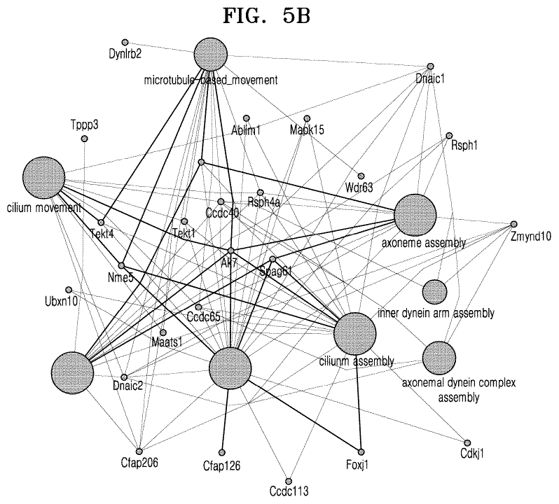

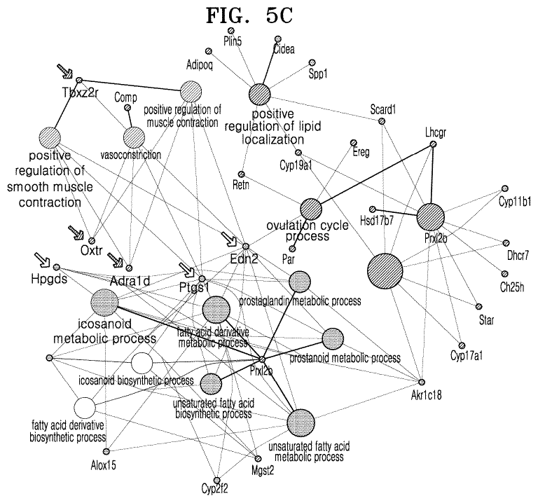

The results of analyzing the entire transcriptomes of the control and the experimental group ovaries through NGS (Next Generation Sequencing) are shown in FIGS. 4 and 5. As shown in FIG. 4, 275 genes showed 4-fold or more of the difference in mRNA expression levels between the RGMc-treated ovaries and the control group. Among the DEGs, the expression of 197 DEGs and 78 DEGs increased in ovaries and control ovaries, respectively (FIGS. 4a and 4b).

In addition, as shown in FIG. 5, in order to more clearly reveal the hyperovulation mechanism of the RGMc-treated ovaries, gene enrichment analysis was performed using ClueGO for the 197 and 78 DEGs, and as a result, among a total of 275 DEGs, 54 DEGs, including 35 up-expressed DGEs and 19 down-expressed DEGs, corresponded to 23 terms (FIG. 5A). Protein-protein interactions between these terms were analyzed using Gene Ontology. To determine key genes, the up-/down-expressed DEGs associated with these terms were plotted together using ClueGO and CluePedia (FIG. 5B). The thick solid line indicates an association based on experimental evidence, the thin solid line indicates an association based on other evidence, and the size and color of the circle indicate the enrichment significance and percentage of up-/down-expressed genes, respectively. It was confirmed that among the 35 up-expressed DEGs, 23 corresponded to 8 terms associated with cilium and microtubule (cilium organization, cilium assembly, microtubule-based movement, microtubule bundle formation, axoneme assembly, axoneme-dynein complex assembly, and inner dynein assembly), and were closely interrelated with each other. (FIG. 5B left). The remaining 12 up-expressed DEGs and a total of 19 down-expressed DEGs corresponded to 7 down-expressed, 7 up-expressed, and 2 non-satisfied terms (FIG. 5B, right).

### Example 5. Analysis of Expression Changes of Prostaglandin and Muscle Contraction-Related Genes According to RGMc Treatment

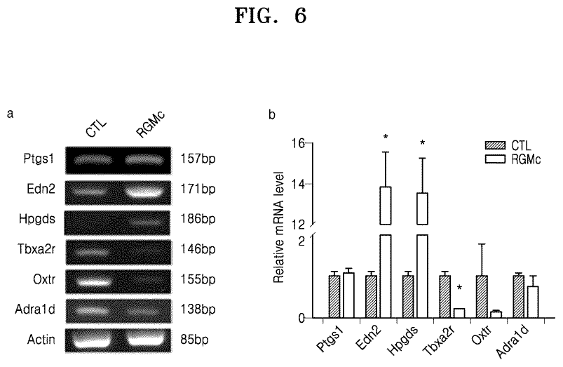

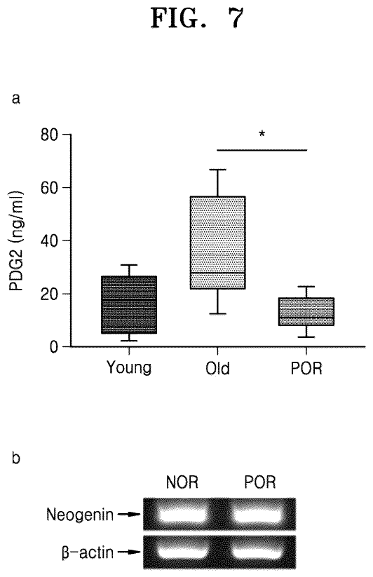

RT-qPCR was performed to experimentally verify the NGS data analyzed in Example 4. As shown in FIG. 6, real-time qPCR was performed on three genes with up-regulated expression levels (FIG. 5B, blue arrows) and three genes with down-regulated expression levels (FIG. 5B, red arrows), and it was confirmed that the mRNA expression levels of Hpgds and Edn2 were significantly increased by 13-fold in RGMc-treated ovaries compared to the control group, while the mRNA expression levels of down-regulated genes such as Tbxa2r were significantly decreased in RGMc-treated ovaries compared to the control group (p<0.05) (FIG. 6A and 6B).

Edn2 is known to be involved in follicle rupture and corpus luteum formation in the ovaries during the reproductive cycle. Additionally, Hpgds interfere with FSH activity in granulosa cells by producing PGD1 and PGD2, and the PGD2 regulates the expression of FSH and LH receptors, and regulates the balance between proliferation, differentiation and steroidogenic activity of granulosa cells in both gonadotropin-independent and gonadotropin-dependent processes of follicular development, and it plays an important role in the continuous ovulatory cascade, including oocyte maturation, cumulus expansion, and follicle maturation. Meanwhile, oxytocin and oxytocin receptor (Oxtr) are associated with POR, and their expression levels are known to be increased in the follicular fluid of patients with polycystic ovarian syndrome (PCOS). However, the role of oxytocin receptors in the relationship with follicular development has not yet been clearly elucidated, and endogenous oxytocin has been reported to be involved in LH regulation in women.

Therefore, the above results confirm that RGMc promotes follicular development through upregulation of Edn2 and Hpgds expression levels, while downregulating the expression levels of genes such as Tbxa2r, thereby being expected to suppress early ovulation and promote normal follicle development.

### Example 6. Analysis of PGD2 Expression Levels in Follicular Fluid and Cumulus Cells

Through the above NGS data, it was confirmed that prostaglandin acts as a key factor in follicular development. Therefore, the relationship between prostaglandins and poor ovarian response (POR) syndrome was analyzed using human follicular fluid and cumulus cells (CC). With approval from the Research Ethics Review Committee of Cha University Medical School, follicular fluid was collected during egg collection, oocytes were removed, and cumulus cells were collected. ELISA was performed using the follicular fluid collected from patients with normal ovarian response and from POR patients with fewer than 4 oocytes and AMH levels of 1 ng/ml or less. The reaction signal was measured with a microplate reader (BioTek Instruments, USA), and gene expression in cumulus cells was analyzed by real-time PCR. Each sample mRNA was isolated from cumulus cells of patients with normal ovarian response and POR patients using a DynaBeads mRNA DIRECT Kit (Life Technologies, Norway). mRNA was quantified using a NanoDrop ND-1000 spectrophotometer (Nyxor Biotech, France). Total RNA was reverse transcribed into cDNA using a AccuPower CycleScript RT PreMix (Bioneer) with poly-dT, and then PCR was performed using the cDNA as a template. Specifically, 10 pmol/μL of forward and reverse primers, 200 ng of template cDNA, and 2× SsoAdvanced Universal SYBR Green Supermix were added to a tube, distilled water was added to be a total volume of 20 μL, and the PCR reaction was performed for a total of 32 cycles, with one cycle of denaturation at 95° C. for 1 minute and at 95° C. for 30 seconds, annealing at 58° C. for 30 seconds, and extension at 72° C. for 50 seconds. After electrophoresis of the PCR product using a 2% agarose gel, the results were determined by UV irradiation using a WSE-6100 LuminoGraph (ATTO, Japan). The primer sequences used are shown in Table 2 below.

TABLE 2

| Gene | Primer sequence (5′-3′) | |
|---|---|---|
| Neogenin | Forward direction | ATTTCCACTCCAGCAGCCTC |
| | (SEQ ID NO: 27) | |
| | Reverse direction | GTGGAATTGGCCCTGTCTGA |
| | (SEQ ID NO: 28) | |
| beta-actin | Forward direction | ACAATGTGCCGAGGACTTT |
| | (SEQ ID NO: 29) | |
| | Reverse direction | TGTGTGGACTTGGGAGAGGA |
| | (SEQ ID NO: 30) | |

### Example 7. Effect of RGMc Treatment on Cumulus Cells

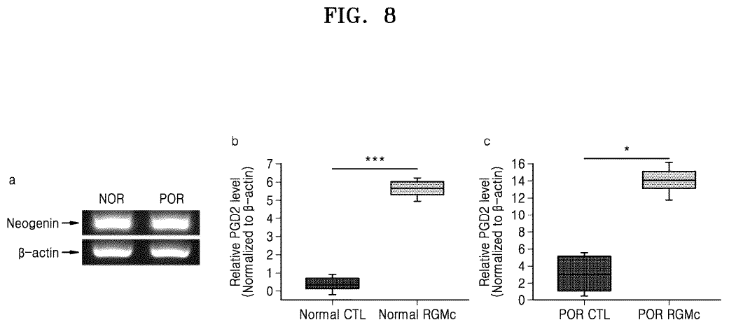

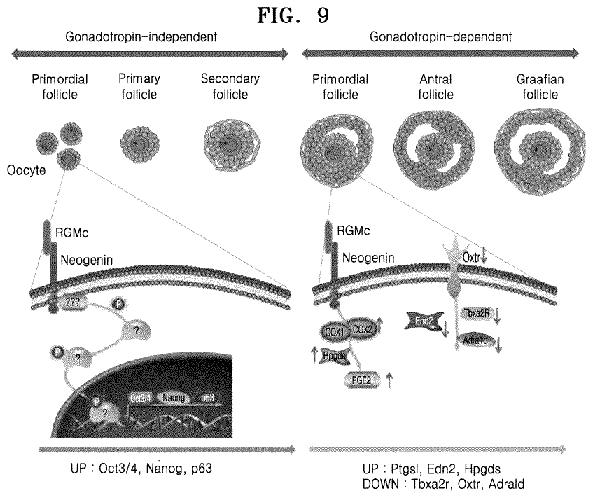

To determine the changes in PGD2 levels according to the treatment of cumulus cells with RGMc, cumulus cells were collected from 8 control patients (normal ovarian response and function) and 8 POR patients (according to Bologna criteria). All patients were subjected to a typical controlled ovarian stimulation (COS) procedure during in vitro fertilization (IVF) by administering a gonadotropin-releasing hormone antagonist. The collected cumulus cells were seeded in a 96-well plate at 10⁴ cells/well in QCL medium. The next day, the culture medium was changed to a medium including 50 ng/ml of RGMc, and after one day, the culture medium was collected and the PGD2 concentration in the medium was measured using an ELISA kit. The results are shown in FIG. 8.

As shown in FIG. 8, it was first confirmed that both neogenin and beta-actin were expressed in the cumulus cells of the control patient and the POR patient (FIG. 8a). Next, it was confirmed that when the cumulus cells collected from the POR patients were treated with RGMc, the level of PGD2 in the cumulus cell culture medium increased significantly by 4-fold (FIG. 8B), and even in patients with normal ovarian response, the level of PGD2 in the cumulus cell culture medium increased following RGMc treatment (FIG. 8C).

Through the above results, it was confirmed that the PGD2 signaling pathway may play a key role in normal follicle development following hormonal stimulation in in vitro fertilization procedures, and the limitations of the COS protocol in existing in vitro fertilization procedures may be overcome through RGMc treatment, and further this may be used to prevent, treat, or ameliorate POR.

### Example 8. Preparation of New Recombinant Protein

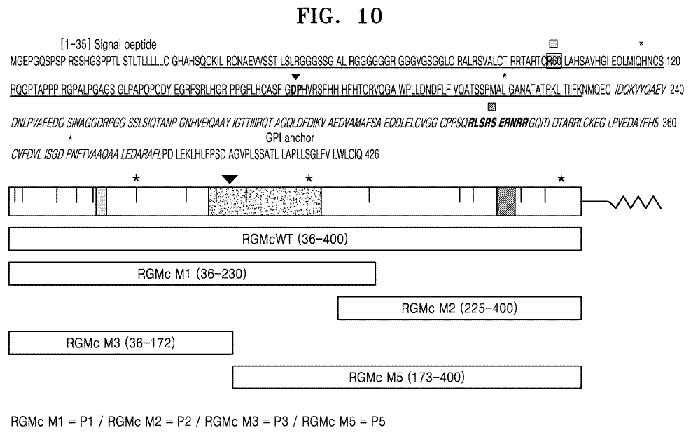

Based on the results of Examples 1 to 7, RGMc recombinant protein with optimal effect was prepared. Specifically, RGMc M1 (P1, SEQ ID NO: 31), RGMc M2 (P2, SEQ ID NO: 32), RGMc M3 (P3, SEQ ID NO: 33) and RGMc M5 (P5, SEQ ID NO: 34), which are recombinant proteins including a specific section other than the signal peptide portion among the entire sequence (426 amino acids) of the human-derived wild-type RGMc protein, were prepared (FIG. 10).

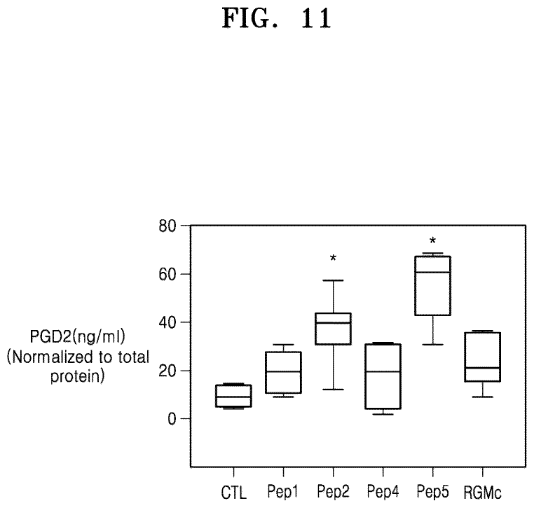

In order to determine the specific effects of the four proteins prepared above, the four proteins and wild-type RGMc were administered to determine the PGD2 expression level. The specific method was carried out according to the method of Example 7. As a result, it was confirmed that the expression of PGD2 increased in all of the groups administered the four newly prepared proteins and RGMc protein, and that among them, PGMc M2 and PGMc M5 may significantly enhance PGD2 (FIG. 11).

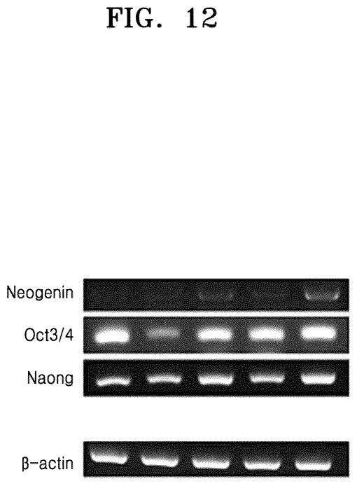

Additionally, an experiment was performed to determine the level of primordial follicle activation of the four proteins prepared above. Specifically, increases and decreases in expression level were determined by comparing Neogenin, Oct3/4, and Nanog with actin, and the method was performed in the same manner as in Example 3. As a result, the activity of primordial follicle factors, especially that of steroid hormone regulatory factors, were significantly increased (FIG. 12).

Through the above results, it was confirmed that among the RGMc-derived proteins, the four newly prepared proteins may activate follicles and prevent, treat, or ameliorate POR. In addition, it was confirmed that among them, PGMc M2 and PGMc M5 showed a more significant effect than the PGMc wild-type, so that they may be useful in preventing, treating or ameliorating POR.

The above description is for illustrative purposes, and those skilled in the art will understand that the present disclosure may be easily modified into other specific forms without changing the technical idea or essential features of the present disclosure. Therefore, the embodiments described above should be understood in all respects as illustrative and not restrictive.

1. A method of preventing or treating poor ovarian response syndrome, comprising:
administering a pharmaceutical composition comprising an RGMc-derived protein.

2. The method of claim 1, wherein the RGMc promotes development of ovarian follicles.

3. The method of claim 1, wherein the RGMc increases expression of one or more selected from the group consisting of Oct3/4, Nanog, and p63.

4. The method of claim 1, wherein the RGMc increases expression of one or more selected from the group consisting of Ptgs1, Edn2, and Hpgds.

5. The method of claim 1, wherein the RGMc reduces expression of one or more selected from the group consisting of Tbxa2r, Oxtr, and Adra1d.

6. The method of claim 1, wherein the RGMc-derived protein is selected from the group comprising amino acid sequences represented by SEQ ID NO: 2, SEQ ID NO: 4, SEQ ID NO: 31, SEQ ID NO: 32, SEQ ID NO: 33, and SEQ ID NO: 34.

7. (canceled)

8. A pharmaceutical composition for preventing, treating or ameliorating infertility, comprising an RGMc-derived protein.

9. The pharmaceutical composition of claim 8, wherein the infertility is infertility due to poor ovarian response syndrome.

10. (canceled)

11. A pharmaceutical composition for promoting pregnancy, comprising an RGMc-derived protein.

12-13. (canceled)


SEQUENCE LISTING (34 sequences)

SEQ ID NO: 1 - DNA, 4054 bp, Mus musculus
  source 1..4054: mol_type = genomic DNA
ccccccacaccaaacctcctctggctctctgacctgagtgagactgcagccattccggggcaatcatggagaaagagatgggggaccccctggctggagcagaccaacagaataggcaactatggctcgagaacccagtatcagagtaatgcttgacctcgggaaacagtaagtccagatgaaatgtcgatcactttgataagcttttgggtcaaggccagaacttcataaagttacagacatctgttctgaaaactaagatctccccttagcaactaccccagtcttcacttttcctcttgcctagatgttttctaatccatggccatttgatgtttaatgctgcctctctctggctttcctattttcctctccatcttattacaccaagaccttgggagagggggagggggcagtgctaaggactgggcccagggccttgagcatggtaaacactcagctaccatcgagttacactcccatccctacctcaagacctctctgacctcctgctcatatgcttatcccaaagtagggttttgacaggagaaaaggagacagaccctcccccatatctgttctcctcaaaagaccaggagtgtctggattgggcttttcagctgggtctggaaaaacacatgaactgttagacagaaacatatattaatttgaatgcttgatttgtaccccatcctgtgagagggatcgtactgtcagccctctggagaggctgagagaccggctctcctttcactgggaaggtgatatcgtagacagggcgtctgcctaccatgttaccctttgtgcccttctaacacagtgaagaaaatgaaacggcaggtggtttctggtgggaaggatccataagaagtatgttcttaaagcgtgatcagatcctggtgctactggggacccactgacatggtcagaacctcactttctctaggggtcctccatttttggcacatctgctatttggtagtcaggatggggcagtacactggactctgcacactgaggtcactcaaccttccctccaccaccaccccgcccccgtaggcatattgcacaagggacctgaaggtggccgcaggggcagggcacagaggctcaccagccacgctcaccctctggaagcctccttcttcccaagatccagtctgtctcatttttgttttggtctcttcagatgctctctctacccaccatcttgactggtctccaacccttctctttccccacactcagccaaatttcttcttccagtcacagaagtacccagagaaattcactaggtaggaggctcatcatctgggaagaaccggtgcctggggggacctggctggataggtatgggccagtcccctagtccccggtccccccacggcagccctccaactctaagcaccctcactctcctgctgctcctctgtggacagggtaaggaagggccagggaagggttggacatatccagaggttaggatgctgggtaggaatgagcgcgcgggggtgggggtgggtgggggtgtgggggtgtggggggtggggggagagtgaggagggatgtgtgctgagtcccagcacctcccaactctgaaagaggcctgtcttgtttcccatttaacttgaagaaggacacagatggcgtccttagaccccagggaaaagaagtctgactgctttacacgtttttagtagaggaaaggagggaaggaggcaaggattgggagaatgaaggttgagattgggaaagcggccacgctgagtggtaggaacggtgggatggcggatggtttttggcagttaggtgcatactggacttggatagaacctgtgtctcatttcactgccctctcccttccgtccccagctcactcccagtgcaagatcctccgctgcaatgccgagtatgtctcgtccactctgagtcttcggggaggtggctcaccggacacgccgcgtggaggcggccgtggtgggctggcctcaggtggcttgtgtcgcgccctgcgctcctacgctctctgcacgcggcgcacggcccgcacctgccgcggggaccttgctttccactctgcggtgcatggcatagaggacctgatgatccagcacaactgctcacgccagggtcccacggccccgcccccggcccggggccccgccctgcccggggccgggccagcgcccctgaccccagatccctgtgactatgaggcccggttttccaggctgcacggtcgagccccgggcttcttgcattgcgcatcctttggagatccccatgtgcgcagtttccacaaccaatttcacacatgccgtgtccaaggagcttggcccttgctagataacgacttcctctttgtccaggccaccagctccccggtttcgtcgggagccaacgctaccaccatccggaaggtcagagattcgattttcatcccaacacaatttcttgagccctctcacggtctgtccgccataccactccttcttcttcccactattcagccacccagctccctgttcttgtctttcccaaacattcccttccatcctgaatggcttccttccatcaaacatttgctcttgtaaatgtaattcaggaatttctttcaaagcaagtaacttgggagttgctgagactgagacaaaaaggagagagagagagggagggagagggagagaggcagatagagagaggcatagggagagagatattaggaggttgagtgggggtcaggcactattaatggaacacagtaggggataggatcaaggactgaggacccggaagaaactcgcatttctgctgggatcggatccctgactctctatgcagcttgattggcgtgaggatgctttgggccctaatgaagtattttctctcttgtcctcatagatcactatcatatttaaaaacatgcaggaatgcattgaccagaaagtctaccaggctgaggtggacaatcttcctgcagcctttgaagatggttctatcaatgggggcgaccgacctgggggctcgagtttgtccattcaaactgctaaccttgggagtcacgtggagattcgagctgcctacattggaacaactatcatcattcgacagacagctgggcagctctccttctccatcagggtagcagaggatgtggcgcgggccttctccgcagagcaggacctacagctgtgtgttgggggatgccctccgagccagcgactctctcgctcagagcgcaaccgccgtggggctatagccatagatactgccagaaggctgtgtaaggaagggcttccggttgaagatgcctacttccaatcctgcgtctttgatgtttcagtctccggtgaccccaactttactgtggcagctcagacagctctggacgatgcccgaatcttcttgacggatttagagaacttacatctctttccctcagatgcggggcctcccctctctcctgccatctgcctagtcccgcttctttcggccctctttgttctgtggctttgcttcagtaagtaggccagcaacccatgactggtttggaaacgatttgaggatagaggttggtgtgagaaaccacaaagatgtgccaaaggaaacagcggggacaggagacaacacttactcaatcagatgaggttgcagtccagggctgaaatgaccctagaataaagattctgggccagggttttgcactccagaccttggtgtgggctattcaccatggatttcccagttagtgatttcccacttgtaatgaaattccactctccatacacctgataccactcctacaagcctagagattgtgagagtgctaatgaccagtgaaacattaaaggactgagatatcgtaaaggcaaaaacatgattctctttgagaaagtcaaaagaggagaagctaattaggaaaagcttttggttcagaaacgaagtgggcattgtctggcagaggaagtcagcttttggagactggcaccaactcagaaacgggcatttccatcccttcctaatctgttattaaagcgattagttctccatcctg

SEQ ID NO: 2 - protein, 420 aa, Mus musculus
MGQSPSPRSPHGSPPTLSTLTLLLLLCGQAHSQCKILRCNAEYVSSTLSLRGGGSPDTPRGGGRGGLASGGLCRALRSYALCTRRTARTCRGDLAFHSAVHGIEDLMIQHNCSRQGPTAPPPARGPALPGAGPAPLTPDPCDYEARFSRLHGRAPGFLHCASFGDPHVRSFHNQFHTCRVQGAWPLLDNDFLFVQATSSPVSSGANATTIRKITIIFKNMQECIDQKVYQAEVDNLPAAFEDGSINGGDRPGGSSLSIQTANLGSHVEIRAAYIGTTIIIRQTAGQLSFSIRVAEDVARAFSAEQDLQLCVGGCPPSQRLSRSERNRRGAIAIDTARRLCKEGLPVEDAYFQSCVFDVSVSGDPNFTVAAQTALDDARIFLTDLENLHLFPSDAGPPLSPAICLVPLLSALFVLWLCFSK

SEQ ID NO: 3 - DNA, 4266 bp, Homo sapiens
  source 1..4266: mol_type = genomic DNA
cttctctggttccctgacctcagtgagacagcagccggcctggggacctgggggagacacggaggaccccctggctggagctgacccacagagtagggaatcatggctggagaattggatagcagagtaatgtttgacctctggaaacagtaagtcaaaatgaaattgcaattcctttaataagcttttatattgaagttagacttttataaaattacaaacacctacttggatgtctctcgtccaaatgctgggatctctccctaccaaggtgccccaatctccatttctctttctgtcttatttctttctggcctctggcctctagctttttgaagtttaattctctgtctctcctctggcagtcttagccctctctttaccttattacctcaagactcctgatgaagttttagaaggagttccctacgtcctctattctgtagttttcttaccaaggccaaatatgacctcagatgatgagtcactgatacccttctatcctgcccccacttagcaatgcccttcacattgagattccaagcatgggggctgctccctgtaaatgatttctccccacaactctagtccctccattctattctccctcttgcaggactcttcccccaatcatatccttacccataagataggggagttaggcaggagggatttagcccctctccaactcctgtcatcataaaagactgagaacttcagaatttgaaaagaagagattaatggaaggagtgatatttgggaaaatacaagaactgttgacttagaaaaaacaaatattgatttgcatgtttggtttgcatcccattattccatgagagagggagattaaaattgcagctctctagagctgatgaaaagagattggtttccttttcatttgaatactgatattctagacgggatgggtatgccacccttaatccttcttgtgttctgacacaaaggaggaaaagaaatgtatgactcctagagggcatctcctcctaatggagagggacaaataagaagtatgtttctgaaatattttcaggtcctaattttactagggtacccactaggattactggtatctgatctagccccatgattcctccatctttgacatacctgctgtttggtagctcagaatggagcaatacagtggactctgcccccttgagttcactcaaccttccctccacccccacctaggggtattgcacaagggccctaaaagtggccacaggaacagggcaaagaggcttaactgccacacttatagtttgaggaactccaatctccccaaattccagtctgttcatccttttcttgatctccccagattcactccacattatccttaccaatcttcaattcttctctctctccatgtccagccaaatttcttttttcagtcacttacagggcttccggtcaaaattcactaggtaggagggtcatcagctgggaagaaccggcgcctgggaaacctggctggataggtatgggggagccaggccagtcccctagtcccaggtcctcccatggcagtcccccaactctaagcactctcactctcctgctgctcctctgtggacatggtaaggaagggccagggaagggtttggggaaatctagagggtaggctgctatgtaggggtgggcatgtgagcctgaatgagtgaggagagataggcgctgagagtcccgatcactcgccctgctctcaaatactaatattttatttcccgttcagtctggggaaggccactggggaagcccttggtcgacaggcagaagagatgtggcaggcttacacacttttagtaagacagccgagagaactagggactagggggttgggggctggggaaggcccttagttaggttttaggaaggctggaaacccctgatgagatttggaagagttatgagcaaactacactccgatagagcagaggtctgaggaccgtctcacaatcctctcccttctgtctttagctcattctcaatgcaagatcctccgctgcaatgctgagtacgtatcgtccactctgagccttagaggtgggggttcatcaggagcacttcgaggaggaggaggaggaggccggggtggaggggtgggctctggcggcctctgtcgagccctccgctcctatgcgctctgcactcggcgcaccgcccgcacctgccgcggggacctcgccttccattcggcggtacatggcatcgaagacctgatgatccagcacaactgctcccgccagggccctacagcccctcccccgccccggggccccgcccttccaggcgcgggctccggcctccctgccccggacccttgtgactatgaaggccggttttcccggctgcatggtcgtcccccggggttcttgcattgcgcttccttcggggacccccatgtgcgcagcttccaccatcactttcacacatgccgtgtccaaggagcttggcctctactggataatgacttcctctttgtccaagccaccagctcccccatggcgttgggggccaacgctaccgccacccggaaggtcaggcactcaatcttccttccgatccacctcatgagattcttccacgggcaccattcctccccatccccactattcaacagcaatgctccctaattcccttttcttcctcaacctctcccccatctcgaatcactcccttctaccaaacacctggagctgtaaatcacttccccttgatgggaatttgactcaaatgcagaaaaccttgaagagacagtcggagagggcggacctgaggagtttcagaagggaaacttttccctctcctaggaagttgccacgattaagtagagagggggttaagtagggatgaggtaatactggaacataaataggagaagggatcaaggattgagggccatagtagtcctgcatctctacttggatcagatctctaactatgtatgaggtctgattggggggaagatgcactgaacccaaaatgaactgttttccctcttgtcctcacagctcaccatcatatttaagaacatgcaggaatgcattgatcagaaggtgtatcaggctgaggtggataatcttcctgtagcctttgaagatggttctatcaatggaggtgaccgacctgggggatccagtttgtcgattcaaactgctaaccctgggaaccatgtggagatccaagctgcctacattggcacaactataatcattcggcagacagctgggcagctctccttctccatcaaggtagcagaggatgtggccatggccttctcagctgaacaggacctgcagctctgtgttggggggtgccctccaagtcagcgactctctcgatcagagcgcaatcgtcggggagctataaccattgatactgccagacggctgtgcaaggaagggcttccagtggaagatgcttacttccattcctgtgtctttgatgttttaatttctggtgatcccaactttaccgtggcagctcaggcagcactggaggatgcccgagccttcctgccagacttagagaagctgcatctcttcccctcagatgctggggttcctctttcctcagcaaccctcttagctccactcctttctgggctctttgttctgtggctttgcattcagtaaggggaccatcagtcccattactagtttggaaatgatttggagatacagattggcatagaagaatgtaaagaatcattaaaggaagcagggcctaggagacacgtgaaacaatgacattatccagagtcagatgaggctgcagtccagggttgaaattatcacagaataaggattctgggcaaggttactgcattccggatctctgtggggctcttcaccaatttttccagcctcatttatagtaaacaaattgttctaatccatttactgcagatttcacccttataagtttagaggtcatgaaggttttaatgatcagtaaagatttaagggttgagatttttaagaggcaagagctgaaagcagaagacatgatcattagccataagaaactcaaaggaggaagacataattagggaaagaagtctatttgatgaatatgtgtgtgtaaggtatgttctgctttcttgattcaaaaatgaagcaggcattgtctagctcttaggtgaagggagtctctgcttttgaagaatggcacaggtaggacagaagtatcatccctaccccctaactaatctgttattaaagctacaaattcttcacacca

SEQ ID NO: 4 - protein, 200 aa, Homo sapiens
MQECIDQKVYQAEVDNLPVAFEDGSINGGDRPGGSSLSIQTANPGNHVEIQAAYIGTTIIIRQTAGQLSFSIKVAEDVAMAFSAEQDLQLCVGGCPPSQRLSRSERNRRGAITIDTARRLCKEGLPVEDAYFHSCVFDVLISGDPNFTVAAQAALEDARAFLPDLEKLHLFPSDAGVPLSSATLLAPLLSGLFVLWLCIQ

SEQ ID NO: 5 - DNA, 20 bp, synthetic construct
  source 1..20: mol_type = other DNA
gtatgtcgcctcgctacctg

SEQ ID NO: 6 - DNA, 20 bp, synthetic construct
  source 1..20: mol_type = other DNA
gccacagagaagtcatcgga

SEQ ID NO: 7 - DNA, 20 bp, synthetic construct
  source 1..20: mol_type = other DNA
cacgagtggaaagcaactca

SEQ ID NO: 8 - DNA, 20 bp, synthetic construct
  source 1..20: mol_type = other DNA
agatggtggtctggctgaac

SEQ ID NO: 9 - DNA, 20 bp, synthetic construct
  source 1..20: mol_type = other DNA
cacccacccatgctagtctt

SEQ ID NO: 10 - DNA, 20 bp, synthetic construct
  source 1..20: mol_type = other DNA
accctcaaactcctggtcct

SEQ ID NO: 11 - DNA, 22 bp, synthetic construct
  source 1..22: mol_type = other DNA
gtatcggacagcgcaaagaacg

SEQ ID NO: 12 - DNA, 22 bp, synthetic construct
  source 1..22: mol_type = other DNA
ctggtaggtacagcagctcatc

SEQ ID NO: 13 - DNA, 20 bp, synthetic construct
  source 1..20: mol_type = other DNA
cctcgacaactaccagtgtg

SEQ ID NO: 14 - DNA, 20 bp, synthetic construct
  source 1..20: mol_type = other DNA
cacaaattcccagagccagt

SEQ ID NO: 15 - DNA, 20 bp, synthetic construct
  source 1..20: mol_type = other DNA
gacctcctccgaaagctgag

SEQ ID NO: 16 - DNA, 20 bp, synthetic construct
  source 1..20: mol_type = other DNA
ccgtttcctcctgtctccac

SEQ ID NO: 17 - DNA, 20 bp, synthetic construct
  source 1..20: mol_type = other DNA
ggacacgctggatgacttca

SEQ ID NO: 18 - DNA, 20 bp, synthetic construct
  source 1..20: mol_type = other DNA
tcccagtagaagtctgccca

SEQ ID NO: 19 - DNA, 20 bp, synthetic construct
  source 1..20: mol_type = other DNA
gttctcaaccatcctcggca

SEQ ID NO: 20 - DNA, 20 bp, synthetic construct
  source 1..20: mol_type = other DNA
ctaaccagcccaaggacagg

SEQ ID NO: 21 - DNA, 20 bp, synthetic construct
  source 1..20: mol_type = other DNA
tcgggctcatattcgcactc

SEQ ID NO: 22 - DNA, 20 bp, synthetic construct
  source 1..20: mol_type = other DNA
aaccatcatctccacctcgc

SEQ ID NO: 23 - DNA, 20 bp, synthetic construct
  source 1..20: mol_type = other DNA
tcttcgtcctgtgctggttc

SEQ ID NO: 24 - DNA, 20 bp, synthetic construct
  source 1..20: mol_type = other DNA
agatgagcgggttcacacag

SEQ ID NO: 25 - DNA, 23 bp, synthetic construct
  source 1..23: mol_type = other DNA
cattgctgacaggatgcagaagg

SEQ ID NO: 26 - DNA, 22 bp, synthetic construct
  source 1..22: mol_type = other DNA
tgctggaaggtggacagtgagg

SEQ ID NO: 27 - DNA, 20 bp, synthetic construct
  source 1..20: mol_type = other DNA
atttccactccagcagcctc

SEQ ID NO: 28 - DNA, 20 bp, synthetic construct
  source 1..20: mol_type = other DNA
gtggaattggccctgtctga

SEQ ID NO: 29 - DNA, 20 bp, synthetic construct
  source 1..20: mol_type = other DNA
acaatgtggccgaggacttt

SEQ ID NO: 30 - DNA, 20 bp, synthetic construct
  source 1..20: mol_type = other DNA
tgtgtggacttgggagagga

SEQ ID NO: 31 - protein, 365 aa, synthetic construct
QCKILRCNAEYVSSTLSLRGGGSSGALRGGGGGGRGGGVGSGGLCRALRSYALCTRRTARTCRGDLAFHSAVHGIEDLMIQHNCSRQGPTAPPPPRGPALPGAGSGLPAPDPCDYEGRFSRLHGRPPGFLHCASFGDPHVRSFHHHFHTCRVQGAWPLLDNDFLFVQATSSPMALGANATATRKLTIIFKNMQECIDQKVYQAEVDNLPVAFEDGSINGGDRPGGSSLSIQTANPGNHVEIQAAYIGTTIIIRQTAGQLSFSIKVAEDVAMAFSAEQDLQLCVGGCPPSQRLSRSERNRRGAITIDTARRLCKEGLPVEDAYFHSCVFDVLISGDPNFTVAAQAALEDARAFLPDLEKLHLFPSD

SEQ ID NO: 32 - protein, 195 aa, synthetic construct
QCKILRCNAEYVSSTLSLRGGGSSGALRGGGGGGRGGGVGSGGLCRALRSYALCTRRTARTCRGDLAFHSAVHGIEDLMIQHNCSRQGPTAPPPPRGPALPGAGSGLPAPDPCDYEGRFSRLHGRPPGFLHCASFGDPHVRSFHHHFHTCRVQGAWPLLDNDFLFVQATSSPMALGANATATRKLTIIFKNMQEC

SEQ ID NO: 33 - protein, 137 aa, synthetic construct
QCKILRCNAEYVSSTLSLRGGGSSGALRGGGGGGRGGGVGSGGLCRALRSYALCTRRTARTCRGDLAFHSAVHGIEDLMIQHNCSRQGPTAPPPPRGPALPGAGSGLPAPDPCDYEGRFSRLHGRPPGFLHCASFGD

SEQ ID NO: 34 - protein, 228 aa, synthetic construct
PHVRSFHHHFHTCRVQGAWPLLDNDFLFVQATSSPMALGANATATRKLTIIFKNMQECIDQKVYQAEVDNLPVAFEDGSINGGDRPGGSSLSIQTANPGNHVEIQAAYIGTTIIIRQTAGQLSFSIKVAEDVAMAFSAEQDLQLCVGGCPPSQRLSRSERNRRGAITIDTARRLCKEGLPVEDAYFHSCVFDVLISGDPNFTVAAQAALEDARAFLPDLEKLHLFPSD